(19) United States
(12) Patent Application Publication
(10) Pub. No.: US 2010/0297041 A1
(43) Pub. Date: Nov. 25, 2010

(54) STABLE DISPERSIONS OF SOLID PARTICLES IN A HYDROPHOBIC SOLVENT AND METHODS OF PREPARING THE SAME

(75) Inventors: Steven Daryl Smith, Fairfield, OH (US); Michael Lee Vatter, Okeana, OH (US); Kevin Lee Doyle, Fairfield, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(73) Assignee: THE PROCTER & GAMBLE COMPANY

(21) Appl. No.: 12/681,952

(22) PCT Filed: Nov. 20, 2008
(86) PCT No.: PCT/US08/84215

§ 371 Date: Aug. 10, 2010

(30) Foreign Application Priority Data
Jun. 15, 2007 (KR) 10-2007-0059174
Feb. 19, 2008 (KR) 10-2008-0014672

(51) Int. Cl. A61K 8/04; A61K 33/26; A61K 33/00; A61Q 19/00; A61Q 17/00; A61Q 1/04; A61Q 1/10; A61K 8/02; A61K 31/74

(52) U.S. Cl. 424/59; 424/646; 424/722; 424/401; 424/78.02

(57) ABSTRACT

A method of dispersing a solid particles in a hydrophobic solvent is disclosed. The method utilizes an acrylate copolymer containing long siloxane chains to provide stable dispersions of solid particles in hydrophobic solvents having a solubility parameter of about 8 or less, such as a silicone fluid.

### FIELD OF THE INVENTION

The present invention relates to stable dispersions of solid particles in a hydrophobic solvent. More particularly, the present invention relates to a stable dispersion of solid particles, such as a pigment, in a hydrophobic solvent having a solubility parameter (γ) of about 8 or less, such as a silicone oil. The present invention also relates to methods of manufacturing the stable dispersions.

### BACKGROUND OF THE INVENTION

It is known to include hydrophobic, i.e., oleophilic, materials, including silicones, in cosmetic products to provide occlusive (moisture retention) properties, and as solvents, for example. It also is known to include solid particles in cosmetic compositions to provide a desired benefit. Depending on the type of solid particles, particle size, and/or configuration, the solid particles can, for example, provide pigmenting and/or sunscreening benefits. In order to provide the benefits of both the oleophilic material and the solid particles, it is reasonable to consider including both of these components in a single composition. This presents difficulties, however, because the solid particles generally do not readily disperse in a hydrophobic matrix or carrier. To overcome this difficulty, solid particles have been stabilized within an oleophilic phase by adding emulsifying agents to a composition.

The steric effect of emulsifiers in a low dielectric constant, i.e. hydrophobic, media may provide particle stabilization and prevent flocculation. In particular, emulsifier molecules coat the free surface of the particle with hydrophilic tails, and oleophilic chains of the surfactant molecule extend into the medium, which prevent agglomeration by osmotic effects, i.e., as two solid particles approach one another, the oleophilic chains overlap and cause a temporary increase in polymer concentration. This concentration increase causes an osmotic stress that forces fluid between the solid particles, thereby causing them to separate.

However, the inclusion of an emulsifier in a composition may not be sufficient to ensure acceptable particle dispersion throughout the life cycle of the product. If the concentration of emulsifier is too low, then the osmotic stress will be correspondingly low and, as two particles approach one another, the emulsifier may bridge the particles to actually promote agglomeration. If, on the other hand, the concentration of emulsifier is too high, then the osmotic stress behavior may be reversed leading to depletion flocculation. In this case, the concentration of free emulsifier may be sufficiently high such that as the particles approach one another, free emulsifier may be forced out from between the particles. This concentration difference may create an osmotic stress that draws fluid out of the space between the particles, thereby promoting agglomeration.

Maintaining a stable dispersion of solid particles in a liquid medium is an important feature of many cosmetic compositions, and the preparation and development of stable dispersions of pigments and sunscreen actives, for example, is an ongoing research endeavor. Stable particle dispersions are desired, and in some cases required, because maximizing particle ability to interact with light and/or the substrate to which the particles are applied is a key to optimizing the performance properties of particles in a composition. It is known that optimizing optical and physical performance requires particles to be stabilized in a deflocculated state. It also is well known that the optical performance of pigment particles in cosmetic products that are not stabilized results in (a) poor shade satisfaction due to color shifts over time, (i.e., the marketed shades do not match the master shade pallet), (b) appearance negatives, such as a cakey appearance due to particle agglomeration in the product during application and drying, (c) poor coverage due to particle agglomeration, and (d) increased manufacturing costs (time required to remediate batches to conform to specifications).

Improved particle dispersion in cosmetic and personal care formulations can lead to an enhanced efficacy for hair and skin care active agents, and to enhanced pigment performance. In particular, superior particle dispersion enhances the optical and physical performance of the particles. Research to improve particle dispersion is directed to developing new raw materials and/or mechanical milling techniques, which are capable of stabilizing and delivering unagglomerated, primary size particles to significantly improve optical properties in liquid make-ups and lipsticks. These optical properties include reduced shade shifting over time (i.e., a reduced ΔE) and stabilized color, improvements in gloss, transparency and chroma, and opacity. Composition esthetics also are improved, e.g., flow because of a lower viscosity and smaller particle size. Wear after application of the composition also is improved because smaller particles are subjected to lower physical abrasion forces and/or electrostatic attachment.

The present invention is directed to providing stable dispersions of solid particles in a hydrophobic liquid, wherein the dispersions provide an enhanced cosmetic benefit.

### SUMMARY OF THE INVENTION

The present invention is directed to stable dispersions of solid particles in a hydrophobic liquid. More particularly, the present invention is directed to a stable dispersion of solid particles, e.g., a pigment or sunscreen active, in a hydrophobic liquid having a solubility parameter (γ) of about 8 or less, for example, a silicone fluid. The present invention also is directed to a method of providing a stable dispersion of solid particles in a hydrophobic liquid.

Accordingly, one aspect of the present invention is to provide a stable dispersion of solid particles in a hydrophobic liquid by utilizing a vinyl block copolymer as a dispersant. The vinyl block copolymers utilized in the present invention are especially effective dispersants for solid particles in a silicone fluid.

Another aspect of the present invention is to provide effective solid particle dispersants for use in a silicone fluid to stabilize primacy particle size cosmetic pigments, which result in increased optical performance, such as improved gloss, opacity, color strength, and transparency.

Another aspect of the present invention is to utilize a vinyl copolymer as a dispersant for solid particles in a hydrophobic medium having a solubility index of about 8 or less, wherein the vinyl copolymer comprises C monomers together with monomers selected from the group consisting of A monomers, B monomers, and mixtures thereof. The vinyl copolymers contain optional A monomers, together with B monomers and C monomers. Preferred vinyl copolymers contain A, B, and C monomers. The vinyl copolymers are described more fully herein, but generally comprise monomers B, C, and optionally, A monomers, which are defined as follows.

Monomer A, when used, comprises at least one free radical polymerizable vinyl monomer. Monomer B comprises at least one monomer copolymerizable with A and C, and selected from the group consisting of polar monomers and macromers having a T_{g} or a Tₘ above about −20° C. Monomer B contains an anchoring group that irreversibly adsorbs to the surface of the solid particles. Monomer, B can be up to about 98%, preferably up to about 90%, 80%, 70%, 60%, 50%, 40%, or 30%, and more preferably up to about 20%, of the total monomers in the copolymer. Monomer C comprises about 0.01% to about 50% of the total monomers in the copolymer.

An anchoring group present in monomer B is selected from the group consisting of carboxyl, carboxylic acid ester, hydroxyl, sulfonate, sulfate, phosphate, phosphonate, nitro, carbohydrate, quaternary ammonium salt, phosphate ester, carbonyl, amino, amide, imide, an aryl group, a heteroaryl group, an aliphatic hydrocarbon, an aliphatic heterocyclic group, polyethylene oxide, polypropylene oxide, silicone, fluorocarbon, polyester, urethane, and mixtures thereof.

Yet another aspect of the present invention is to provide a stable dispersion of solid particles in a hydrophobic medium that is suitable for personal care and cosmetic applications, the stable dispersion comprising:

(a) about 0.5% to about 85%, by weight, solid particles, such as a pigment;

(b) about 0.01% to about 60%, by weight, of a vinyl copolymer; and

(c) about 10% to about 90%, by weight, of a hydrophobic solvent, such as a silicone fluid.

These and other novel aspects of the present invention will become apparent from the following detailed descriptions of the preferred embodiments.

### BRIEF DESCRIPTION OF THE PREFERRED EMBODIMENTS

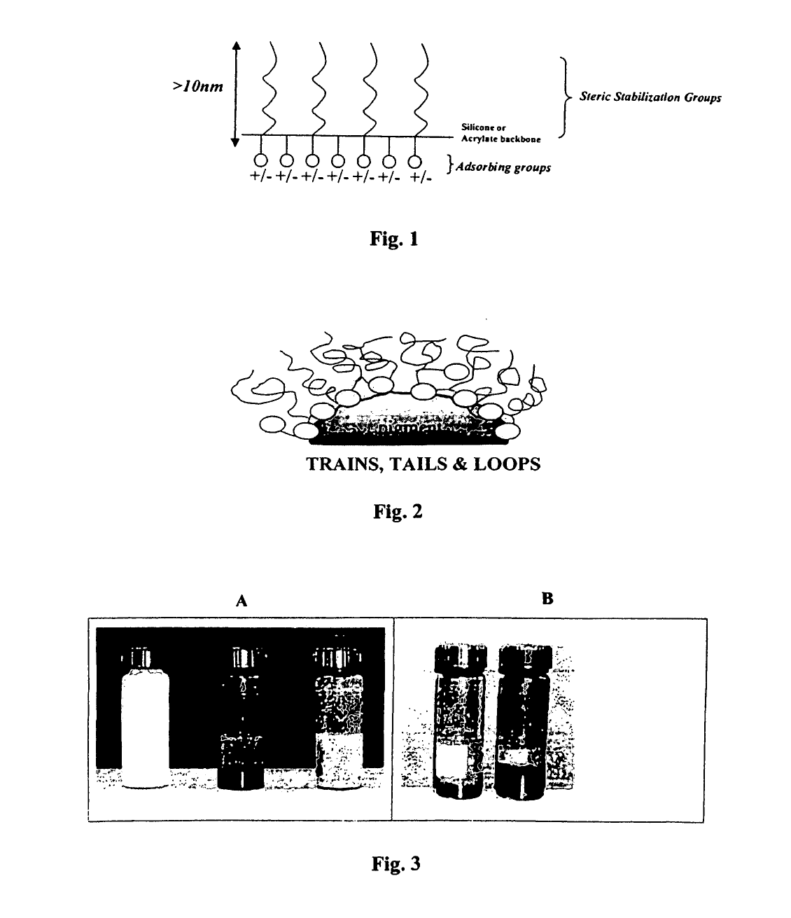

FIG. 1 is a schematic of a vinyl copolymer utilized in the present invention;

FIG. 2 is a schematic showing the interaction between a solid particle and a vinyl copolymer; and

FIG. 3 are photographs illustrating stable dispersions provided by the present invention.

### DETAILED DESCRIPTION OF THE PREFERRED EMBODIMENTS

All weights, measurements, and concentrations herein are measured at 25° C., unless otherwise specified.

Unless otherwise indicated, all percentages of compositions and ingredients referred to herein are weight percentages of the total composition (i.e., the sum of all components present) and ratios are weight ratios.

Unless otherwise indicated, all polymer molecular weights are number average molecular weights.

Reference herein to the percentage weight of a compound in a composition is the percentage weight of the compound in that composition, not to the percentage weight of the compound plus solvent in the composition. This is stated for the purposes of clarity, because commercially available compounds often are sold as less than 100% active in combination with a solvent or other diluent.

Unless otherwise indicated, all patents and publications referred to herein are incorporated by reference.

Except where specific examples of actual measured values are presented, numerical values referred to herein should be considered to be qualified by the word “about”.

Historically, pigments and other solid particles have been dispersed in cosmetic formulations by the use of nonionic silicone and hydrocarbon surfactants, or by surface treatments, in an attempt to stabilize the particles in a given composition. However, presently-used surfactants to stabilize pigments do not strongly absorb to particle surfaces to prevent particle flocculation and surface treatments only improve particle wetting. The disadvantages associated with the surfactant and surface treatment approaches have resulted in poorly stabilized solid particles which flocculate in the composition to produce large particles exhibiting low gloss, reduced opacity, and poor color strength.

The dispersants disclosed herein provide solid particle dispersions that demonstrate improved optical performance, i.e., improved gloss, transparency, color strength, coverage/opacity, improved feel, and wear. Testing also shows that particle dispersions prepared using a vinyl copolymer disclosed herein exhibit the best color strength observed to date for silicone oil-based cosmetic compositions.

Without being bound to any theory, it is believed that the vinyl copolymers irreversibly adsorb to the surface of the solid particles via an anchoring group, such as amino or carboxylate moieties, and sterically stabilize the particles in a hydrophobic liquid, like a silicone fluid, via long siloxane chains present in the vinyl copolymer.

A present stabilized dispersion of solid particles comprises:

(a) about 0.5% to about 85%, by weight, of solid particles;

(b) about 0.01% to about 60%, by weight, of a vinyl copolymer; and

(c) about 10% to about 90%, by weight, of a hydrophobic solvent.

The stabilized dispersions can further contain optional ingredients commonly used in cosmetic and personal care products.

### Solid Particles

The solid particles present in the stabilized composition are insoluble in the hydrophobic liquid, and impart a benefit after application to skin and/or hair. The solid particles are nanometer and micrometer in size, and an important aspect of the present invention is to reduce, minimize, or eliminate agglomeration of the small primary particles to form large secondary particles, which may not impart the desired benefit to skin or hair. The solid particles can be, for example, platelet shaped, spherical, elongated or needle-shaped, or irregularly shaped; surface coated or uncoated; porous or non-porous; charged or uncharged; and can be added to the current compositions as a powder or as a pre-dispersion.

Typically, the solid particle is a pigment, i.e., a colorant, that is insoluble in a hydrophobic liquid having a solubility parameter (γ) of about 8 or less. It should be noted that a pigment is different from a dye, which is a colorant that is soluble in a hydrophobic liquid having a solubility parameter of about 8 or less.

As used herein, the term “pigment” includes true pigments (i.e., colorants that precipitate as they are manufactured), toners (i.e., colorants produced by precipitating a water soluble dye as a metal salt), and lakes (i.e., nonsoluble colorants produced by adsorbing a water soluble dye onto an insoluble substrate).

The solid particles included in a present stabilized dispersion are not limited to pigments, but also can be other solid particles that impart a benefit to treated skin or hair, like a feel or friction reducing agent, sunscreen agent or skin care agent.

Solid particles having a small particle size possess a highly reactive surface that can cause undesired chemical or photochemical reactions. To avoid this problem, it is known to dope surfaces of small particles with one or more known doping materials, such as silica or a metal oxide, like alumina, to reduce the reactivity of the particle surface. This surface treatment typically comprises about 15% to about 30%, by weight of the solid particle. Solid particles used in a present stabilized dispersion therefore can be doped or undoped.

The solid particles of the stabilized dispersion have a number weighted average particle size of about 10 nm (nanometers) to about 100 um (micrometers), preferably about 15 nm to about 10 um, more preferably about 20 nm to about 1 um, yet more preferably about 25 to about 40 nm As used herein, the number weighed average particle is determined by x-ray diffraction, based on measuring the broadening of the strongest rutile line, or by using a Nicomp 370 Sub Micron Particle Sizer.

Stabilized dispersions according to the present invention comprise about 0.5 wt % to 90 wt % solid particles, preferably about 5 wt % to about 80 wt %, and more preferably about 15 wt % to about 70 wt % solid particles.

Examples of solid particles that can be dispersed by the vinyl copolymer include, but are not limited to, aluminum powder, bismuth oxychloride, bronze powder, chlorophyllin-copper complex, chromium hydroxide green, chromium oxide greens, copper powder, D & C Blue No. 1 Aluminum Lake, D & C Green No. 3 Aluminum Lake, D & C Orange No. 4 Aluminum Lake, D & C Orange No. 5 Aluminum Lake, D & C Orange No. 5 Zirconium Lake, D & C Orange No. 10 Aluminum Lake, D & C Orange No. 17 Lake, D & C Red No. 3 Aluminum Lake, D & C Red No. 4 Aluminum Lake, D & C Red No. 6 Aluminum Lake, D & C Red No. 6 Barium Lake, D & C Red No. 6 Barium/Strontium Lake, D & C Red No. 6 Potassium Lake, D & C Red No. 6 Strontium Lake, D & C Red No. 7 Aluminum Lake, D & C Red No. 7 Barium Lake, D & C Red No. 7 Calcium Lake, D & C Red No. 7 Calcium/Strontium Lake, D & C Red No. 7 Zirconium Lake, D & C Red No. 8 Sodium Lake, D & C Red No. 9 Aluminum Lake, D & C Red No. 9 Barium Lake, D & C Red No. 9 Barium/Strontium Lake, D & C Red No. 9 Zirconium Lake, D & C Red No. 19 Aluminum Lake, D & C Red No. 19 Barium Lake, D & C Red No. 19 Zirconium Lake, D & C Red No. 21 Aluminum Lake, D & C Red No. 21 Zirconium Lake, D & C Red No. 27 Aluminum Lake, D & C Red No. 27 Barium Lake, D & C Red No. 27 Calcium Lake, D & C Red No. 27 Zirconium Lake, Red 28 Aluminum Lake, D & C Red No. 30 Lake, D & C Red No. 31 Calcium Lake, D & C Red No. 33 Aluminum Lake, D & C Red No. 34 Calcium Lake, D & C Red No. 36 Lake, D & C Red No. 40 Aluminum Lake, D & C Yellow No. 5 Aluminum Lake, D & C Yellow No. 5 Zirconium Lake, D & C Yellow No. 6 Aluminum Lake, D & C Yellow No. 7 Zirconium Lake, D & C Yellow No. 10 Aluminum Lake, Ext. D & C Yellow No. 7 Aluminum Lake, FD & C Blue No. 1 Aluminum Lake, FD & C Red No. 3 Aluminum Lake, FD & C Red No. 4 Aluminum Lake, FD & C Yellow No. 5 Aluminum Lake, FD & C Yellow No. 6 Aluminum Lake, iron oxides, mica, silver, titanium dioxide, zinc oxide, zinc sulfide, lithopane (a mixture of zinc sulfide and barium sulfate), carbon black, and mixtures thereof.

There are no specific limitations as to the solid particles, e.g., pigment, colorant, lake dyes, toner, or filler powders, used in the composition. The solid particles can be scattering or non-scattering, and may or may not impart color. The solid particles can be natural, synthetic, or semi-synthetic in composition. Hybrid particles are also useful. Synthetic particles can be made of either crosslinked or non-crosslinked polymers. The particles of the present invention can have surface charges or their surface can be modified with organic or inorganic materials such as surfactants, polymers, and inorganic materials. Particle complexes are also useful. Each may be a body pigment, inorganic white pigment, inorganic colored pigment, pearling agent, and the like.

Specific examples include, but are not limited to, inorganic powders such as gums, chalk, Fullers earth, kaolin, sericite, muscovite, phlogopite, synthetic mica, lepidolite, biotite, lithia mica, vermiculite, aluminum silicate, starch, smectite clays, alkyl and/or trialkyl aryl ammonium smectites, aluminum magnesium silicate, chemically-modified magnesium aluminum silicate, organically modified montmorillonite clay, hydrated aluminum silicate, fumed aluminum starch octenyl succinate, barium silicate, calcium silicate, magnesium succinate, barium silicate, calcium silicate, magnesium silicate, strontium silicate, metal tungstate, magnesium silica, alumina, zeolite, barium sulfate, calcined calcium sulfate (calcined gypsum), calcium phosphate, fluorine apatite, hydroxyapatite, ceramic powder, metallic soap (e.g., zinc stearate, magnesium stearate, zinc myristate, calcium palmitate, and aluminum stearate), colloidal silicone dioxide, and boron nitride; an organic powder, such as polyamide resin powder (nylon powder), cyclodextrin, copolymer powder of styrene and acrylic acid, benzoguanamine resin powder, carboxyvinyl polymer, cellulose powder, such as hydroxyethyl cellulose and sodium carboxymethyl cellulose, ethylene glycol monostearate; inorganic white pigments, such as magnesium oxide, talc, mica, magnesium carbonate, calcium carbonate, magnesium silicate, silica, titanium dioxide, zinc oxide, red iron oxide, yellow iron oxide, black iron oxide, ultramarine, silk powder, crystalline cellulose, starch, titanated mica, iron oxide titanated mica, bismuth oxychloride, and the like, including mixtures thereof. Other useful solid particles are disclosed in U.S. Pat. No. 5,688,831, incorporated herein by reference. These solid particles can be used independently or in combination.

Additional suitable particles include fumed silica, spherical silica, micronized teflon, acrylate polymers, bentonite, corn starch, diatomaceous earth, glyceryl starch, hectorite, hydrated silica, magnesium hydroxide, magnesium oxide, magnesium trisilicate, maltodextrin, montmorillonite, microcrystalline cellulose, rice starch, zinc laurate, zinc neodecanoate, zinc rosinate, attapulgite, dextran, silica silylate, soy flour, tin oxide, titanium hydroxide, trimagnesium phosphate, walnut shell powder, or mixtures thereof. The above mentioned particles may be surface treated with lecithin, collagen, amino acids, mineral oil, silicone oil, metal soaps, alkyl silane, isotitanium triisostearate, PEG siloxanes, fluorosiloxane, fluorinated materials, polyethylene, magnesium myristate, lysine derivatives, active plant lipid extracts or various other agents either alone or in combination, which coat the powder surface and render the particles hydrophobic in nature.

Non-limiting examples of natural particles include various precipitated silica particles in hydrophilic and hydrophobic forms available from Degussa-Huls under the trade name SIPERNET. Snowtex colloidal silica particles, available from Nissan Chemical America Corporation, also are useful.

Examples of synthetic particles which can improve skin and hair feel (feel modifier) include nylon, silicone resins, poly(meth)acrylates, polyethylene, polyester, polypropylene, polystyrene, polyurethane, polyamide, epoxy resins, urea resins, and acrylic powders. Non-limiting examples of useful particles are Microease 1105, 114S, 116 (micronized synthetic waxes), Micropoly 210, 2505 (micronized polyethylene), Microslip (micronized polytetrafluoroethylene), and Microsilk (combination of polyethylene and polytetrafluoroethylene), all of which are available from Micro Powder, Inc. Other examples include Luna (smooth silica particles) particles available from Phenomenex, MP-2200 (polymethyl methacrylate), EA-209 (ethylene/acrylate copolymer), SP-501(nylon-12), ES-830 (polymethyl methacrylate), BPD-800, BPD-500 (polyurethane) particles available from Kobo Products, Inc. and silicone resins sold under the name Tospearl particles by GE Silicones. Ganzpearl GS-0605 crosslinked polystyrene (available from Presperse) is also useful.

Non-limiting examples of hybrid particles include Ganzpearl GSC-30SR (sericite & crosslinked polystyrene hybrid powder), and SM-1000, SM-200 (mica and silica hybrid powder available from Presperse).

Cosmetic compositions based on a stabilized dispersion of the present invention typically comprise metal oxide particles, which can be particles of any suitable metal oxide. Preferably, the metal oxide particles are selected from the group consisting of titanium oxide, zinc oxide, zirconium oxide, yellow iron oxide, black iron oxide, tin oxide, red iron oxide, chromium oxide, chromium hydroxide, cerium oxide, zinc cerium oxide, and mixtures thereof. More preferably, the metal oxide particles are selected from the group consisting of titanium dioxide, zinc oxide, and mixtures thereof. More preferably still, the metal oxide particles comprise titanium dioxide particles.

In addition to colorants, the solid particles can be an active sunscreen compound and/or an ultraviolet light absorber. The active sunscreen compound can be any compound that protects the skin from the effects of the sun and that is insoluble in a hydrophobic solvent. Examples of active sunscreen compounds, in addition to the colorants and metal oxides disclosed above, include, but are not limited to both sunscreen agents and physical sunblocks, organic or inorganic, or disclosed in The Cosmetic, Toiletry, and Fragrance Association's The International Cosmetic Ingredient Dictionary and Handbook, 10^{th} Ed., Gottschalck et al., Eds. (2004), p. 2267 and pp. 2292-93. Particularly suitable sunscreen compounds include, but are not limited to, benzophenone, benzophenone-1, benzophenone-2, benzophenone-3, benzophenone-4, benzophenone-5, benzophenone-6, benzophenone-7, benzophenone-8, benzophenone-9, benzophenone-10, benzophenone-11, benzophenone-12, benzotriazolyl dodecyl p-cresol, 3-benzylidene camphor, benzylidene camphor sulfonic acid, benzyl salicylate, bis-ethylhexyloxyphenol methoxyphenyl triazine, bornelone, bumetrizole, butyl methoxydibenzoylmethane, butyl PABA (p-aminobenzoic acid), cinnamidopropyl trimonium chloride, cinoxate, deamethoxycinnamate, dibenzoxazoyl naphthalene, di-t-butyl hydroxybenzylidene camphor, diethylamino hydroxybenzoyl hexyl benzoate, diethylhexyl butamido triazone, diethylhexyl 2,6-naphthalate, diisopropyl ethyl cinnamate, diisopropyl methyl cinnamate, dimethoxycinnamidopropyl ethyldimonium chloride ether, dimethyl PABA, ethyl cetearyldimonium tosylate, dimorpholinopyridazinone, dimorpholinopyridazinone, disodium bisethylphenyl triaminotriazine stilbenedisulfonate, disodium distrylbiphenyl disulfonate, disodium phenyl dibenzimidazole tetrasulfonate, drometrizole, drometrizole trisiloxane, ethyl dihydroxypropyl PABA, ethyl diisopropyl-cinnamate, ethylhexyl bis-isopentylbenzoxazolylphenyl melamine, ethyl dimethoxybenzylidene dioxoimidazolidine propionate, ethylhexyl dimethyl PABA, ethylhexyl methoxycinnamate, ethylhexyl methoxydibenzoylmethane, ethylhexyl salicylate, ethylhexyl triazone, ethyl methoxycinnamate, ethyl PABA, ethyl urocanate, etocrylene, 4-(2-beta-glucopyranosiloxy)propoxy-2-hydroxybenzophenone, glyceryl ethylhexanoate dimethoxycinnamate, glyceryl PABA, glycol salicylate, hexanediol disalicylate, homosalate, isoamyl cinnamate, isoamyl p-methoxycinnamate, isopentyl trimethoxycinnamate trisiloxane, isopropylbenzyl salicylate, isopropyl dibenzoylmethane, isopropyl methoxycinnamate, kaempferia glaga root extract, menthyl anthranilate, menthyl salicylate, methoxycinnamidopropyl hydroxy sulaine, methoxycinnamidopropyl laurdimonium tosylate, 4-methylbenzylidene camphor, methylene bis-benzotriazolyl tetramethylbutyl-phenol, octocrylene, octrizole, PABA, PEG-25 PABA, phenylbenzimidazole sulfonic acid, polyacrylamidomethyl benzylidene camphor, polyamide-2, polyquaternium-59, polysilicone-15, potassium methoxycinnamate, potassium phenylbenzimidazole sulfonate, red petrolatum, sodium benzotrizoyl butylphenol sulfonate, sodium phenylbenzimidazole sulfonate, sodium urocanate, TEA-phenylbenzimidazole sulfonate, TEA-salicylate, terephthalyidene dicamphor sulfonic acid, tetrabutyl phenyl hydroxybenzoate, and urocanic acid.

In addition to pigments and active sunscreen compounds, the solid particles can be, for example, an opacifier; a skin, hair, or nail care active; filler; a feel modifier; or any solid particle suitable for inclusion in a cosmetic or personal care composition, and that is insoluble in a hydrophobic solvent.

The solid particles therefore can be, for example, one or more vitamin. Herein, “vitamins” means vitamins, pro-vitamins, and their salts, isomers and derivatives. Non-limiting examples of suitable vitamins include: vitamin B compounds (including B1 compounds, B2 compounds, and B3 compounds, such as niacinamide, niacinnicotinic acid, tocopheryl nicotinate, C1-C18 nicotinic acid esters, and nicotinyl alcohol; B5 compounds, such as panthenol or “pro-B5”, panthothenic acid, pantothenyl; B6 compounds, such as pyroxidine, pyridoxal, pyridoxamine; carnitine, thiamine, riboflavin); vitamin A compounds, and all natural and/or synthetic analogs of Vitamin A, including retinoids, retinol, retinyl acetate, retinyl palmitate, retinoic acid, retinaldehyde, retinyl propionate, carotenoids (pro-vitamin A), and other compounds which possess the biological activity of Vitamin A; vitamin D compounds; vitamin K compounds; vitamin E compounds; or tocopherol, including tocopherol sorbate, tocopherol acetate, other esters of tocopherol and tocopheryl compounds; vitamin C compounds, including ascorbate, ascorbyl esters of fatty acids, and ascorbic acid derivatives, for example, ascorbyl phosphates, such as magnesium ascorbyl phosphate and sodium ascorbyl phosphate, ascorbyl glucoside, and ascorbyl sorbate; and vitamin F compounds, such as saturated and/or unsaturated fatty acids. In one embodiment, the composition comprises a vitamin selected from the group consisting of vitamin B compounds, vitamin C compounds, vitamin E compounds, and mixtures thereof. Alternatively, the vitamin is selected from the group consisting of niacinamide, tocopheryl nicotinate, pyroxidine, panthenol, vitamin E, vitamin E acetate, ascorbyl phosphate, ascorbyl glucoside, and mixtures thereof.

The solid particles also can comprise one or more peptides. Herein, “peptide” refers to peptides containing ten or fewer amino acids, their derivatives, isomers, and complexes with other species such as metal ions (for example, copper, zinc, manganese, and magnesium). As used herein, peptide refers to both naturally occurring and synthesized peptides. In one embodiment, the peptides are di-, tri-, tetra-, penta-, and hexa-peptides, their salts, isomers, derivatives, and mixtures thereof. Examples of useful peptide derivatives include, but are not limited to, peptides derived from soy proteins, carnosine (beta-alanine-histidine), palmitoyl-lysine-threonine (pal-KT) and palmitoyl-lysine-threonine-threonine-lysine-serine (pal-KTTKS, available in a composition known as MATRIXYL®), palmitoyl-glycine-glutamine-proline-arginine (pal-GQPR, available in a composition known as RIGIN®), these three being available from Sederma, France, acetyl-glutamate-glutamate-methionine-glutamine-arginine-arginine (Ac-EEMQRR; ARGIRELINE®), and Cu-histidine-glycine-glycine (Cu-HGG, also known as IAMIN®).

The solid particles also can comprise a sugar amine, also known as amino sugars, and their salts, isomers, tautomers, and derivatives. Sugar amines can be synthetic or natural in origin and can be used as pure compounds or as mixture of compounds (e.g., extracts from natural sources or mixtures of synthetic materials). For example, glucosamine is generally found in many shellfish and can also be derived from fungal sources. Sugar amine compounds useful in the present invention include, for example, N-acetyl-glucosamine, and also those described in PCT Publication WO 02/076423 and U.S. Pat. No. 6,159,485.

The solid particles also can comprise a flavonoid. The flavonoid can be a synthetic material or an extract from natural sources, which can be further derivatized. Examples of classes of suitable flavonoids are disclosed in U.S. Pat. No. 6,235,773, and include, but are not limited to, unsubstituted favanones, methoxy flavanones, unsubstituted chalcones, and mixtures thereof. In one embodiment, the flavonoids are unsubstituted flavanones, unsubstituted chalcone (especially the trans-isomer), their glucosyl derivatives, and mixtures thereof. Other examples of suitable flavonoids include flavanones such as hesperidin and glucosyl hesperidin, isoflavones, such as soy isoflavones, including but not limited to genistein, daidzein, quecetin, and equol, their glucosyl derivatives, 2′,4-dihydroxy chalcone, and mixtures thereof.

The solid particles further can comprise non-vitamin antioxidants and radical scavengers, minerals, preservatives, phytosterols and/or plant hormones, protease inhibitors, tyrosinase inhibitors, and anti-inflammatory agents.

Non-vitamin antioxidants and radical scavengers include, but are not limited to, BHT (butylated hydroxy toluene), butylated hydroxy benzoic acids, L-ergothioneine (available as THIOTANE™), tetrahydrocurcumin, cetyl pyridinium chloride, diethylhexyl syrinylidene malonate (available as OXYNEX™), 6-hydroxy-2,5,7,8-tetramethylchroman-2-carboxylic acid (available as TROLOX™), hexadec-8-ene-1,16-dicarboxylic acid (octadecene dioic acid; available as ARLATONE™ Dioic DCA from Uniqema), ubiquinone (co-enzyme Q10), tea extracts, including green tea extract, yeast extracts or yeast culture fluid (e.g., PITERA™), gallic acid, uric acid, sorbic acid, lipoic acid, amines (e.g., N,N-diethylhydroxylamine, aminoguanidine), sulfhydryl compounds, including glutathione, dihydroxy fumaric acid, lycine pidolate, arginine pilolate, nordihydroguaiaretic acid, curcumin, lysine, methionine, proline, superoxide dimutase, silymarin, grape skin/seed extracts, melanin, rosemary extracts, salts and derivatives of any of the foregoing, and combinations thereof.

The solid particles also can be a mineral, plant sterol, protein inhibitor, tyrosinase inhibitor, and anti-inflammatory agent.

Minerals include, but are not limited to, zinc, manganese, magnesium, copper, iron, selenium and other mineral supplements. “Mineral” is understood to include minerals in various oxidation states, mineral complexes, salts, derivatives, and combinations thereof.

Plant sterols (phytosterols) and/or plant hormones include, but are not limited to, sitosterol, stigmasterol, campesterol, barssicasterol, kinetin, zeatin, and derivatives and mixtures thereof.

Protease inhibitors include, but are not limited to, hexamidine, vanillin acetate, menthyl anthranilate, soybean trypsin inhibitor, Bowman-Birk inhibitor, and mixtures thereof.

Tyrosinase inhibitors include, but are not limited to, sinablanca (mustard seed extract), tetrahydrocurcumin, cetyl pyridinium chloride, and mixtures thereof.

Anti-inflammatory agents include, but are not limited to nonsteroidal antiinflammatory agents (NSAIDS), including but not limited to ibuprofen, naproxen, flufenamic acid, etofenamate, aspirin, mefenamic acid, meclofenamic acid, piroxicam and felbinac; glycyrrhizic acid (also known as glycyrrhizin, glycyrrhixinic acid, and glycyrrhetinic acid glycoside), glycyrrhetenic acid, other licorice extracts; candelilla wax, bisabolol (e.g., alpha bisabolol), manjistha (extracted from plants in the genus Rubia, particularly Rubia cordifolia), and guggal (extracted from plants in the genus Commiphora, particularly Commiphora mukul), kola extract, chamomile, red clover extract, and sea whip extract, derivatives of any of the foregoing, and mixtures thereof.

Other useful skin cares actives that can be the solid particles include moisturizing and/or conditioning agents, such as allantoin, bisabolol, dipotassium glycyrrhizinate, and urea; dehydroepiandrosterone (DHEA), its analogs and derivatives; exfoliating agents, including alpha- and beta-hydroxyacids, alpha-keto acids, glycolic acid and octanoyl salicylate; desquamation actives, including zwitterionic surfactants; antimicrobial agents; anti-cellulite agents, such as caffeine, theophylline, theobromine, and aminophylline; antidandruff agents such as piroctone olamine, 3,4,4′-trichlorocarbanilide (triclosan), triclocarban, and zinc pyrithione; dimethyl aminoethanol (DMAE); creatine; plant-derived materials such as resveratrol; chelators, for example, furildioxime and furilmonoxime; dialkanoyl hydroxyproline compounds; soy extracts, such as soybean milk, soybean paste, and miso salts; amino acids; topical anaesthetics, such as benzocaine, lidocaine, bupivacaine, chlorprocaine, dibucaine, etidocaine, mepivacaine, tetracaine, dyclonine, hexylcaine, procaine, cocain, ketamine, pramoxine, phenol; salts and derivatives of any of the foregoing; and mixtures thereof.

The solid particles used in the composition can be hollow particles. In a preferred embodiment, the hollow particles are fluid-encapsulated, flexible microspheres. The microspheres are structurally hollow, however, they may contain various fluids, which encompass liquids and gases and their isomers. The gases include, but not limited to, butane, pentane, air, nitrogen, oxygen, carbon dioxide, and dimethyl ether. If used, liquids may only partially fill the microspheres. The liquids include water and any compatible solvent. The liquids may also contain vitamins, amino acids, proteins and protein derivatives, herbal extracts, pigments, dyes, antimicrobial agents, chelating agents, UV absorbers, optical brighteners, silicone compounds, perfumes, humectants which are generally water soluble, additional conditioning agents which are generally water insoluble, and mixtures thereof. In one embodiment, water soluble components are preferred encompassed material. In another embodiment, components selected from the group consisting of vitamins, amino acids, proteins, protein derivatives, herbal extracts, and mixtures thereof are preferred encompassed material. In yet another embodiment, components selected from the group consisting of vitamin E, pantothenyl ethyl ether, panthenol, Polygonum multiflori extracts, and mixtures thereof are preferred encompassed material.

The solid particles can have surface charges or their surface can be modified with organic or inorganic materials such as surfactants, polymers, and inorganic materials. Particle complexes are also useful. Non-limiting examples of complexes of gas-encapsulated microspheres are DSPCS-I2™ (silica modified ethylene/methacrylate copolymer microsphere) and SPCAT-I2™ (talc modified ethylene/methacrylate copolymer microsphere). Both of these are available from Kobo Products, Inc.

The surface of the solid particles may be charged through a static development or with the attachment of various ionic groups directly or linked via short, long or branched alkyl groups. The surface charge can be anionic, cationic, zwitterionic or amphoteric in nature.

Solid particles comprised of polymers and copolymers obtained from esters, such as, for example, vinyl acetate or lactate, or acids, such as, for example, itaconic, citraconic, maleic or fumaric acids may also be used. See Japanese Patent Application No. JP-A-2-112304, the disclosure of which is incorporated herein by reference.

Non-limiting examples of commercially available suitable particles are 551 DE (particle size range of approximately 30-50 um and density of approximately 42 kg/m³), 551 DE 20 (particle size range of approximately 15-25 um and density of approximately 60 kg/m³), 461 DE (particle size range of approximately 20-40 um and density 60 kg/m³), 551 DE 80 (particle size of approximately 50-80 um and density of approximately 42 kg/m³), 091 DE (particle size range of approximately 35-55 um and density of approximately 30 kg/m³), all of which are marketed under the trademark EXPANCEL™ by Akzo Nobel. Other examples of suitable particles for use herein are marketed under the trademarks DUALITE® and MICROPEARL™ series of microspheres from Pierce & Stevens Corporation. Particularly preferred hollow particles are 091 DE and 551DE 50. The hollow particles of the present invention exist in either dry or hydrated state. The aforesaid particles are nontoxic and nonirritating to the skin.

Hollow solid particles can be prepared, for example, via the processes described in EP 56,219, EP 348,372, EP 486,080, EP 320,473, EP 112,807 and U.S. Pat. No. 3,615,972, the disclosure of each of which is incorporated herein by reference.

### Vinyl Copolymer

The dispersion of solid particles in the hydrophobic solvent is stabilized by the addition of about 0.01% to about 200%, by weight, of a vinyl copolymer, based on the weight and overall surface area of the solids in the dispersion. Preferably, the vinyl copolymer is present in an amount of about 0.01% to about 80%, and more preferably about 0.05% to about 60%, by weight of the dispersion. Typically the amount of vinyl copolymer needed corresponds to 2 mg dispersant/cm² of particle surface area.

A vinyl copolymer useful in the present invention has a molecular weight of 15,000 or greater, and is not crosslinked. The vinyl copolymer contains a plurality of hydrophobic tails greater than about 10 nm in length. The acrylate copolymer also has a silicone and an optional acrylate backbone, and a plurality of groups that anchor the vinyl copolymer to a solid particle. The anchoring group is selected from the group consisting of carboxyl, carboxylic acid ester, hydroxyl, sulfonate, sulfate, phosphate, phosphonate, nitro, carbohydrate, quaternary ammonium salt, phosphate ester, carbonyl, amino, amide, imide, an aryl group, a heteroaryl group, an aliphatic hydrocarbon, an aliphatic heterocyclic group, polyethylene oxide, polypropylene oxide, silicone, fluorocarbon, polyester, urethane, and mixtures thereof. The anchoring, or absorbing, group preferably is a hydrophilic binding species, such as carboxyl and amino moieties. The vinyl copolymer typically contains about 7 or more adsorbing groups. FIG. 1 is a schematic representation of a vinyl copolymer adsorbed onto a solid particle, with the hydrophobic tails extending into a hydrophobic medium.

In its broadest aspect, the vinyl copolymers utilized in the present invention comprise C monomers together with monomers selected from the group consisting of B monomers and optional A monomers. The vinyl copolymers contain C monomers together with B monomers and optional A monomers, and preferred copolymers contain A, B, and C monomers.

Examples of useful copolymers and their method of manufacture are described in detail in U.S. Pat. Nos. 4,693,935; 4,728,571; 5,100,658; 5,106,609; 5,277,899; and 5,565,193, each incorporated herein by reference. These copolymers comprise monomers C, B, and optionally, A, which are defined as follows. A, when used, is at least one free radically polymerizable vinyl monomer. B comprises at least one reinforcing monomer copolymerizable with A and is selected from the group consisting of polar monomers and macromers having a Tg or a Tm above about −20° C. B may be up to about 98%, preferably up to about 90%, 80%, 70%, 60%, 50%, 30%, or 25%, more preferably up to about 20%, of the total monomers in the copolymer. Monomer C comprises from about 0.01% to about 50% of the total monomers in the copolymer.

Representative examples of A monomers are acrylic or methacrylic acid esters of C₁-C₁₈ alcohols, i.e., alcohols having one to about 18 carbon atoms with the average number of carbon atoms being from about 4-12; such as methanol, ethanol, 1-propanol, 2-propanol, 1-butanol, 2-methyl-1-propanol, 1-pentanol, 2-pentanol, 3-pentanol, 2-methyl-1-butanol, 1-methyl-1-butanol, 3-methyl-1-butanol, 1-methyl-1-pentanol, 2-methyl-1-pentanol, 3-methyl-1-pentanol, t-butanol, cyclohexanol, 2-ethyl-1-butanol, 3-heptanol, benzyl alcohol, 2-octanol, 6-methyl-1-heptanol, 2-ethyl-1-hexanol, 3,5-dimethyl-1-hexanol, 3,5,5-trimethyl-1-hexanol, 1-decanol, 1-dodecanol, 1-hexadecanol, 1-octadecanol, and the like. Other useful A monomers include, but are not limited to, styrene; vinyl acetate; vinyl chloride; vinylidene chloride; acrylonitrile; alpha-methylstyrene; t-butylstyrene; butadiene; cyclohexadiene; ethylene; propylene; vinyl toluene; and mixtures thereof. Preferred A monomers include n-butyl methacrylate, isobutyl methacrylate, 2-ethylhexyl methacrylate, methyl methacrylate, t-butyl acrylate, t-butyl methacrylate, and mixtures thereof.

Representative examples of B monomers include acrylic acid, methacrylic acid, N,N-dimethylacrylamide, dimethylaminoethyl methacrylate, quaternized dimethylaminoethyl methacrylate, methacrylonitrile, polystyrene macromer, methacrylamide, maleic anhydride and its half esters, itaconic acid, acrylamide, acrylate alcohols, hydroxyethyl methacrylate, diallyldimethyl ammonium chloride, vinyl pyrrolidone, vinyl ethers (such as methyl vinyl ether), maleimides, vinyl pyridine, vinyl imidazole, other polar vinyl heterocyclics, styrene sulfonate, and mixtures thereof. Preferred B monomers include acrylic acid, N,N-dimethylacrylamide, dimethylaminoethyl methacrylate, quaternized dimethylaminoethyl methacrylate, vinyl pyrrolidone, and mixtures thereof.

The C monomer has a general formula:

X(Y)ₙSi(R)₃₋ₘZₘ,

wherein X is a vinyl group copolymerizable with the A and B monomers; Y is a divalent linking group; R is hydrogen, lower alkyl, aryl, or alkoxy; Z is a monovalent siloxane polymeric moiety having a number average molecular weight of at least about 500, is essentially unreactive under copolymerization conditions, and is pendant from the vinyl polymeric backbone, described above; n is 0 or 1; and m is an integer from 1 to 3. C has a weight average molecular weight of from about 1,000 to about 50,000, preferably from about 5,000 to about 40,000, most preferably from about 10,000 to about 20,000. Preferably, the C monomer has a formula selected from the following group:

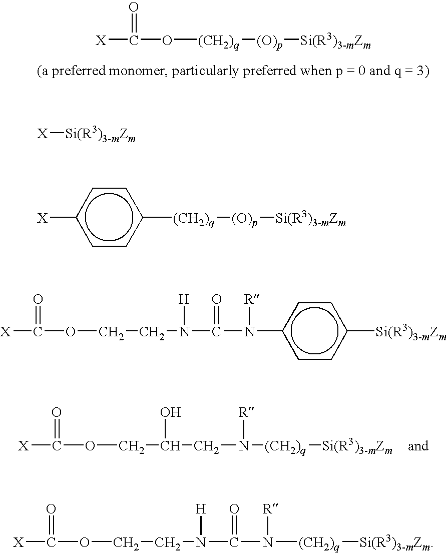

In the above structures, m is 1, 2 or 3 (preferably m=1); p is 0 or 1; R″ is alkyl or hydrogen; q is an integer from 2 to 6; X is

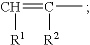

R¹ is hydrogen or —COOH (preferably R¹ is hydrogen); R² is hydrogen, methyl, or —CH₂COOH (preferably R² is methyl); Z is

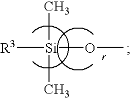

R³ is alkyl, alkoxy, alkylamino, aryl, or hydroxyl (preferably R⁴ is alkyl); and r is an integer from about 5 to about 700 (preferably r is about 200 to about 300).

Preferred acrylate polymers useful in the present invention generally comprise 0% to about 98% (preferably about 5% to about 98%, more preferably about 50% to about 90%) of monomer A, about 0.1% to about 98% (preferably about 7.5% to about 80%) of monomer B, and about 0.1% to about 50% (preferably about 0.5% to about 40%, most preferably about 2% to about 25%) of monomer C. The combination of the A and B monomers preferably comprises about 50% to about 99.9% (more preferably about 60% to about 99%, most preferably from about 75% to about 95%) of the copolymer. The composition of any particular copolymer helps determine its formulation properties.

The following are nonlimiting examples of a class of vinyl copolymers (I) useful in the present invention

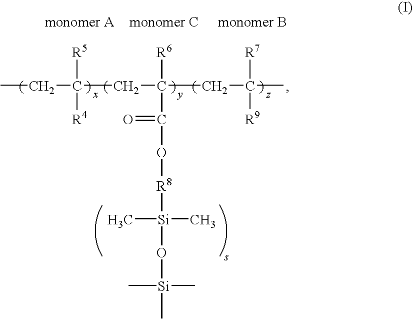

wherein R⁴ is selected from the group consisting of —C(═O)R^{a}, —C(═O)OR^{a}, and —C(═O)NR^{a}R^{b};

R⁵ is selected from the group consisting of C₁₋₃alkyl, aryl, and —C(═O)R^{a};

R⁶ is selected from the group consisting of C₁₋₃alkyl, aryl, and —C(═O)R^{a};

R⁷ is selected from the group consisting of C₁₋₃alkyl, aryl, and —C(═O)R^{a};

R⁸ is CH₂₁₋₆;

R⁹, independently, is selected from the group consisting of carboxyl, carboxylic acid ester, hydroxyl, sulfonate, sulfate, phosphate, phosphonate, nitro, carbohydrate, quaternary ammonium salt, phosphate ester, carbonyl, amino, amide, imide, an aryl group, a heteroaryl group, an aliphatic hydrocarbon, an aliphatic heterocyclic group, polyethylene oxide, polypropylene oxide, silicone, fluorocarbon, polyester, and urethane;

s is an integer from 1 to 500;

R^{a} and R^{b}, independently, are C₁₋₁₂ alkyl or aryl;

x is an integer 0 to 500;

y is an integer 2 to 25; and

z is an integer 1 to 500.

In preferred embodiments of vinyl copolymer (I), R⁴ is —C(═O)R^{a}, R⁵, R⁶, and R⁷, independently, are
C₁₋₃alkyl, aryl, and —C(═O)R^{a}; R⁸ is CH₂₁₋₆; R⁹ is amino or carboxy; and R^{a} and R^{b} are C₁₋₃alkyl.

As used herein, the term “alkyl” includes straight chained and branched hydrocarbon groups containing the indicated number of carbon atoms, e.g., C₁₋₁₂, like methyl, ethyl, propyl, and butyl groups.

The term “aryl,” alone or in combination, is defined herein as a monocyclic or bicyclic aromatic group, e.g., phenyl or naphthyl. Unless otherwise indicated, an “aryl” group can be unsubstituted or substituted, for example, with one or more, and in particular one to three, halo, alkyl, hydroxyalkyl, alkoxy, alkoxyalkyl, haloalkyl, nitro, amino, alkylamino, acylamino, alkylthio, alkylsulfinyl, and alkylsulfonyl.

In other preferred embodiments, the vinyl copolymer (I) has a structure (II) or (III)

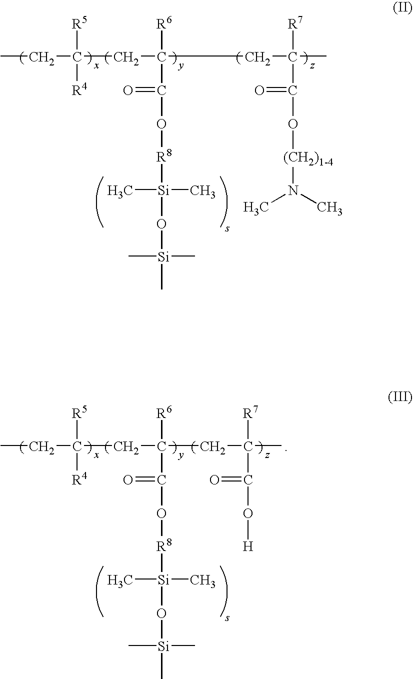

The following are nonlimiting examples of an amino-containing vinyl copolymer (IIa) and a carboxyl-containing vinyl copolymer (IIIa). The positively-charged amino groups of (IIa) and the negatively-charged carboxyl groups of (IIIa) allow adsorption, or anchoring, of the vinyl polymer onto surfaces of the solid particles.

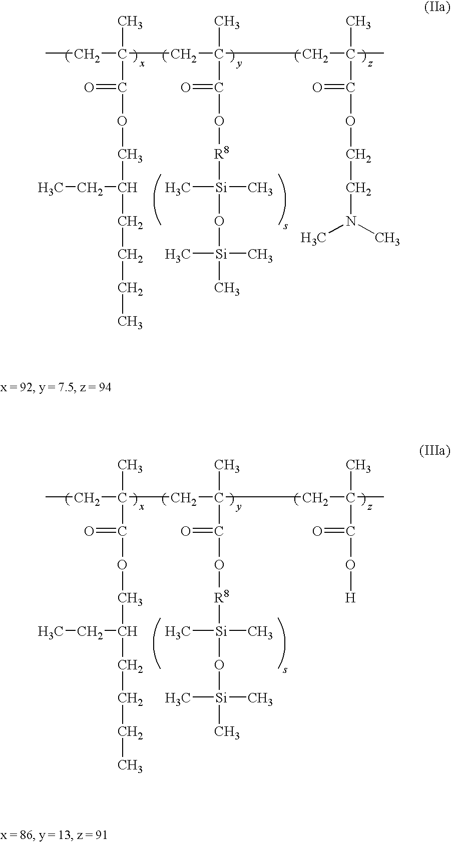

Exemplary polymers for use in the present invention include the following (the weight percents below refer to the weight amount of monomers added in the polymerization reaction, not necessarily the amount in the finished polymer):
acrylic acid/n-butyl methacrylate/polydimethylsiloxane (PDMS) macromer—20,000 molecular weight (10/70/20 w/w/w) (MW—100,000),
N,N-dimethylacrylamide/isobutyl methacrylate/PDMS macromer—20,000 molecular weight (20/60/20 w/w/w),
dimethylaminoethyl methacrylate/isobutyl methacrylate/2-ethylhexyl methacrylate/PDMS macromer—20,000 molecular weight (25/40/15/20 w/w/w/w),
dimethylacrylamide/PDMS macromer—20,000 molecular weight (80/20 w/w),
t-butyl acrylate/t-butyl methacrylate/PDMS macromer—10,000 molecular weight (56/24/20 w/w/w),
t-butyl acrylate/PDMS macromer—10,000 molecular weight (80/20 w/w),
t-butyl acrylate/N,N-dimethylacrylamide/PDMS macromer—10,000 molecular weight (70/10/20 w/w/w),
t-butyl acrylate/acrylic acid/PDMS macromer-10,000 molecular weight (75/5/20 w/w/w).
N,N-dimethylaminoethyl methacrylate/isobutyl methacrylate/PDMS macromer—20,000 molecular weight (10/70/20 w/w/w) (MW—400,000),
quaternized N,N-dimethylaminoethyl methacrylate/isobutyl methacrylate/PDMS macromer—20,000 molecular weight (60/20/20 w/w/w) (MW—500,000),
acrylic acid/methyl methacrylate/PDMS macromer—20,000 molecular weight (40/40/20 w/w/w) (MW—400,000),
acrylic acid/n-butyl methacrylate/PDMS macromer—20,000 molecular weight (10/70/20 w/w/w) (MW—300,000),
acrylic acid/isopropyl methacrylate/PDMS macromer—10,000 molecular weight (25/65/10 w/w/w) (MW—200,000),
N,N-dimethylacrylamide/methoxyethyl methacrylate/PDMS macromer—20,000 molecular weight (60/25/15 w/w/w) (MW—200,000),
N,N-dimethylacrylamide/isobutyl methacrylate/2-ethylhexyl methacrylate/PDMS macromer—20,000 molecular weight (12/64/4/20 w/w/w) (MW—300,000),
N,N-dimethylaminoethyl methacrylate/isobutyl methacrylate/2-ethylhexyl methacrylate/PDMS macromer—20,000 molecular weight (30/40/10/20 w/w/w/w) (MW—300,000), and
t-butyl acrylate/PDMS macromer—10,000 molecular weight (80/20 w/w) (MW—150,000)

The PDMS macromer of molecular weight of about 20,000 is prepared in a manner similar to Example C-2c of U.S. Pat. No. 4,728,571, and the PDMS of molecular weight of about 10,000 is prepared in a manner similar to Example C-2b of U.S. Pat. No. 4,728,571, incorporated herein by reference.

### Hydrophobic Solvent

The solid particles are dispersed in a hydrophobic solvent having a solubility parameter of about 8 or less. The solubility parameter (γ) is determined as set forth in “Solubility-Effects in Product, Package, and Preservation,” C. D. Vaughn, Cosmetics and Toiletries, vol. 103, pages 60-62, 64 (1988).

The following are exemplary hydrophobic solvents wherein the acrylate copolymer effectively disperses the solid particles to provide a stable dispersion:

| Hydrophobic Solvent | Solubility Parameter (γ) |
|---|---|
| Perfluorohexane | 5.68 |
| Perfluoroctane | 5.72 |
| Cyclomethicone D5 | 5.77 |
| Dimethicone | 5.92 |
| Cyclomethicone D4 | 5.99 |
| Squalane | 6.03 |
| Hexamethyldisiloxane | 6.15 |
| Isocetyl Stearate | 6.19 |
| Perfluorodecalin | 6.34 |
| Neopentane | 6.38 |
| Safflower Oil | 6.42 |
| Melene (C30) | 6.58 |
| Docosane (C22) | 6.60 |
| Almond Oil | 6.81 |
| Isopentane | 6.82 |
| Avocado Oil | 6.83 |
| Nonacosane (C29) | 6.83 |
| Arachidic Acid | 6.85 |
| Pristane | 6.85 |
| Decyl Oleate | 6.92 |
| C8-Isoparaffin | 6.93 |
| Diisopropyl Ether | 6.95 |
| Sperm Oil | 7.09 |
| White Mineral Oil | 7.09 |
| Pentane | 7.10 |
| Tricosane (C22) | 7.13 |
| Isodecyl Oleate | 7.17 |
| Cholesteryl Oleate | 7.24 |
| Peanut Oil | 7.24 |
| Hexane | 7.28 |
| Linseed Oil | 7.29 |
| Octadecane (C18) | 7.29 |
| Cyclohexane | 7.30 |
| Dioctyl Ether | 7.30 |
| Eicosane (C20) | 7.32 |
| Lanolin Oil | 7.33 |
| Petrolatum | 7.33 |
| Behenic Acid | 7.35 |
| Diethyl Ether | 7.37 |
| Corn Oil-Refined | 7.40 |
| Cetane (C16) | 7.41 |
| Heptane | 7.41 |
| Isostearyl Neopentanoate | 7.43 |
| Octyl Palmitate | 7.44 |
| Propyl Fluroide | 7.48 |
| Rice Oil - SO | 7.48 |
| Tridecane (C13) | 7.48 |
| Propellant 11 | 7.49 |
| Cottonseed Oil | 7.52 |
| C12-15 Alcohols Benzoate | 7.53 |
| Isopropyl Linoleate | 7.55 |
| Cod Liver Oil | 7.56 |
| Erucic Acid | 7.57 |
| Butyl Stearate | 7.58 |
| Octane | 7.58 |
| Cetyl Octanoate | 7.59 |
| Decene-1 | 7.59 |
| Dodecane | 7.59 |
| Diethylhexyl Adipate | 7.60 |
| Decane | 7.62 |
| Isobutyl Stearate | 7.65 |
| Butyl Myristate | 7.68 |
| Stearic Acid (C18) | 7.74 |
| Dioctyl Maleate | 7.75 |
| Octyl Fluoride | 7.76 |
| Isopropyl Palmitate | 7.78 |
| Dioctyl Adipate | 7.82 |
| Oleth-3 | 7.83 |
| Diethylamine | 7.86 |
| Linolenic Acid | 7.86 |
| Olive Oil | 7.87 |
| Palmitic Acid (C16) | 7.89 |
| Oleic Acid | 7.91 |
| PEG-4 Stearate | 7.92 |
| Tetraethyl Lead | 7.92 |
| Tridecyl Neopentanoate | 7.92 |
| Pentaerythritol Tetraoleate | 7.98 |
| Tocopheryl Acetate | 7.98 |
| Ethyl Myristate | 8.00 |
| Isopropyl Myristate | 8.02 |
| Turpentine (pinene) | 8.03 |
| Human Erythrocyte | 8.05 |
| Methyl Oleate | 8.05 |
| Cetyl Acetate | 8.06 |
| Methyl Linoleate | 8.08 |
| Isotearic Acid | 8.09 |
| Coconut Oil | 8.10 |
| Myristic Acid (C14) | 8.10 |

Additional useful hydrophobic solvents include, but are not limited to, isododecane, volatile and non-volatile silicone fluids (10 centistoke (cSt) through 1000 cSt, for example), isononyl isononanoate, heptanoic acid esterified with 2,2-dimethyl-1,3-propenediol and isododecane (LEXFEEL D4 and D5), isodecyl neopentanoate, isotridecyl isononoate, C₅₋₉ carboxylic acids tetraesters with pentaerythritol and C₅₋₉ carboxylic acids hexaesters with dipentaerythritol (LEXFEEL 70), ABIL EM90 polysiloxane, octyldodecyl stearate, DC5225C silicone, octyldodecyl neopentanoate, and heptanoic acid esterified with 2,2-dimethyl-1.3-propenedial (LEXFEEL 7), trimethylolpropane tricaprylate/tricaprate (LEXFEEL 21), dipentaerythrityl hexa C₅₋₉ acid esters (LEXFEEL 350), propylene glycol dibenzoate (LEXFEEL SHINE), DC556 silicone, and decanoic acid esterified with 2-ethyl-2-(hydroxymethyl)-1,3-propenediol octanolate.

The above exemplary hydrophobic solvents can be used individually, or in any mixture of two or more of the above solvents. In addition, the hydrophobic solvents can be admixed with a solvent having a solubility parameter (γ) of greater than about 8, as long as the resulting solvent has a solubility parameter of less than about 8.

To demonstrate the new and unexpected benefits provided by the present invention, dispersants (i.e., vinyl copolymers) and hydrophobic solvents were screened for an ability to provide stable non-aqueous dispersions of solid particles. Vinyl copolymer compatibility in the hydrophobic solvent void of solid particles is based on the appearance and physical properties of the solvent, i.e., the hydrophobic solvent is not affected by the addition of the dispersant (5-10% w/w). The copolymer is either completely soluble or dispersable in the hydrophobic solvent

In testing compositions for dispersion stability, shaker bottles were prepared as follows:

In a 4 oz glass bottle add:

a) 4.5 g solid particles, 2.0 g dispersant, 40.0 g hydrophobic solvent, 30.0 g sand or other grinding media.

b) Shake the resulting mixture on a Red Devil Shaker for about 20-30 minutes, then remove the bottles.

c) Visually observe the shaken bottle over time.

Settling of the sand and some solid particles will occur. However, if the bottles remain cloudy over time, with a slow persistent settling, and microscopy reveals considerable Brownian motion, with no flocks, then an interaction between the dispersant and the surface of the solid particle has occurred. If no interaction occurs, flocks of soft voluminous bulk pigment quickly result, and the dispersant can be eliminated as a potential particle-stabilizing candidate. At high solids levels (>70%) flocculation can be induced by the addition of too much dispersant. For particles that do not interact with a dispersant, the solvent system can be changed to improve wetting.

The recommended level of dispersant is 2 mg/m² of particle surface. Based on the above general procedures, screening of dispersants and solvents showed that an excellent dispersant systems for solid particles comprises the combination of an vinyl copolymer and a hydrophobic solvent having a solubility parameter of less than about 8. In particular, the settling test showed that both the amine and carboxyl functionalized vinyl copolymers strongly absorbed onto surfaces of the solid particles, and prevented settling of the solid particles (e.g., titanium dioxide, red, yellow, and black iron oxide) tested in cyclomethicone D5 hydrophobic solvent. An amine-functionalized acrylate copolymer surprisingly outperformed all dispersant polymers tested for a tested black iron oxide.

In particular, FIG. 3 contains photographs of vials containing compositions comprising solid particles, a dispersant, and a hydrophobic solvent. The compositions were tested and observed for dispersion stability. Each vial of FIG. 3a contains a mixture of 5.88% wt. solid particles (titanium dioxide, red iron oxide, yellow iron oxide, or black iron oxide), 52.29% wt. Cyclopentasiloxane/D5, an acrylate copolymer (2.61% wt.), and 39.22% wt. grinding media (⅛″ MR-8640 stainless steel balls from BYK Gardner). The mixture was shaken on a Red Devil Paint Shaker for 30 minutes, then allowed to stand to observe pigment settling. If most of the solid particles remain suspended over time, the adsorption/interaction of the acrylate copolymer on the pigment was demonstrated. Minimal settling of the titanium dioxide, red iron oxide, and yellow iron oxide was observed over time, indicating that the vinyl copolymer adsorbed onto the solid particle surfaces to provide steric stabilization and prevent settling. For the black iron oxide samples, the vial on the right of FIG. 3b contains an amine-functionalized vinyl copolymer and the vial on the left contains a carboxyl functionalized acrylate copolymer. Even though there is some settling of the black iron oxide in both vials, the height of the layer on the right vial indicates that the amine-functionalized vinyl copolymer adsorbed and provided some particle spacing via steric stabilization.

The following are nonlimiting examples of compositions of the present invention.

### Example 1

A moisturizing lotion of the present invention is prepared as follows:

| Ingredient | Wt % |
|---|---|
| Cyclomethicone (DC245) | 17.35 |
| DC9040 Elastomer Gel² | 18.0 |
| Elastomer Gel (KSG210)¹ | 18.33 |
| Propylparaben | 0.2 |
| Ethylene/Acrylic Acid Copolymer microspheres | 7.0 |
| (Flobeads EA 209 supplied by Kobo Products Inc.) | |
| Vinyl Block Copolymer (dispersant) | 1.0 |
| Pigment | 2.0 |
| Glycerin | 25.0 |
| Water | 8.0 |
| Skin care actives | 3.0 |
| Methylparaben | 0.12 |
| Total | 100.0 |

¹25% Dimethicone/Copolyol Crosspolymer in dimethicone;
²12% Dimethicone/Vinyl Dimethicone crosspolymer (aver. particle size of at least 20 microns) in cyclomethicone

In a suitable stainless steel vessel, the vinyl block copolymer is dissolved or dispersed in the cyclomethicone solvent. The pigment and microspheres are added, and high shear milled using conventional milling techniques (typically 0.3-0.4 mm zirconium coated ceramic beads are used in a media mill) until desired particle size is achieved. The DC9040 (nonemulsifying silicone elastomer), KSG210 (emulsifying silicone elastomer), and propylparaben then are added with mixing using conventional mixing technology and mixed until homogeneous. In a separate vessel, the skin care actives, water, glycerin, and methylparaben are mixed until homogeneous. Next, this polar phase mixture is admixed with the cyclomethicone pigment dispersion using conventional mixing technology until homogeneous. The resulting mixture then is poured into suitable containers. The moisturizing cosmetic lotion is applied to the face and/or body to provide softening, moisturization, and conditioning.

### Example 2

A liquid foundation of the present invention is prepared as follows:

| Ingredient | Wt % |
|---|---|
| Cyclomethicone (DC245) | 11.62 |
| Vinyl Block Copolymer | 1.0 |
| Dimethicone Copolyol emulsifier | 0.7 |
| KSG32 Elastomer Gel¹ | 5.38 |
| GE SFE839 Elastomer Gel² | 10.0 |
| Isononyl Isononanoate | 4.0 |
| n-Propyl-4-hydroxybenzoic Acid | 0.20 |
| Ethylene Brassylate | 0.03 |
| Titanium Dioxide | 17.8 |
| Yellow Iron Oxide | 1.70 |
| Red Iron Oxide | 0.19 |
| Black Iron Oxide | 0.11 |
| Methyl Parahydroxybenzoate | 0.12 |
| Glycerin | 10.0 |
| Water | 36.45 |
| Sucrose oleate ester | 0.6 |
| Total | 100.00 |

¹25%% Lauryl Dimethicone/Copolyol Crosspolymer in isododecane;
²5% Dimethicone/Vinyl Dimethicone crosspolymer (aver. particle size of at least 20 microns) in cyclomethicone

In a suitable stainless steel vessel, the vinyl block copolymer is dispersed in the cyclomethicone, then the pigments (titanium dioxide and iron oxides) are added and milled using conventional milling techniques, such as media milling or high shear milling, until the desired particle size. Then, the dimethicone copolyol, GE SFE 839, KSG32, isononyl isononanoate, n-propyl-4-hydroxybenzoic acid, and ethylene brassylate are added with mixing using conventional mixing technology and mixed until homogeneous. In a separate vessel equipped with a heat source, the sucrose oleate ester, water, glycerin, and methyl parahydroxybenzoate are heated to 50° C. and mixed using conventional mixing technology until homogeneous. The sucrose oleate ester mixture then is allowed to cool to room temperature. Once cooled, the sucrose oleate ester mixture is combined with the cyclomethicone mixture and mixed using conventional mixing technology until homogeneous. The combined mixture then is poured into suitable containers. The liquid foundation is applied to the face to provide softening, moisturization, and conditioning.

### Example 3

A line-minimizing product that improves the appearance of skin texture is prepared as follows:

| Ingredient | Wt % |
|---|---|
| DC9040 Cross Linked elastomer Gel¹ | 50.25 |
| Elastomer gel (KSG 210)² | 5.00 |
| Cyclomethicone (SF1202) | 10.00 |
| Vinyl Block Copolymer | 1.25 |
| Silica, Titanium Dioxide, Iron Oxide | 8.00 |
| (Ronasphere LDP) | |
| Isoeicosane (Permethyl 102A) | 5.00 |
| Alkyl Methicone (DC AMS C30 wax) | 1.50 |
| Propylparabens | 0.25 |
| Tocopherol Acetate | 0.50 |
| Water | 9.35 |
| Glycerin | 5.75 |
| Niacinamide | 2.00 |
| Panthenol | 0.50 |
| Sodium Dehydroacetate | 0.30 |
| Disodium EDTA | 0.10 |
| Phenoxyethanol | 0.25 |

¹12% Dimethicone/Vinyl Dimethicone crosspolymer (aver. particle size of at least 20 microns) in cyclomethicone;
²25% Dimethicone/Copolyol Crosspolymer in dimethicone

In a suitable vessel, add the water, glycerin, niacinamide, panthenol, sodium dehydroacetate, disodium EDTA, and phenoxyethanol. The resulting mixture is mixed using conventional technology until a clear water phase is achieved. In a separate vessel equipped with a heating source, the AMS wax and Permethyl are added and heated to 75° C. with gentle mixing. In a third vessel, the vinyl block copolymer is dissolved in the cyclomethicone (DC245), and the Ronasphere is added while mixing to form the Ronasphere/DC245 pre-mix. Once the wax/Permethyl mixture is fully molten, the DC9040 and KSG21 elastomers are added and this mixture is mixed until homogeneous. The wax/Permethyl/elastomer mixture is mixed using a Heidolph overhead stirrer (Model #RZR50), or equivalent, on low speed (about 50-100 rpm) while cooling the mixture to room temperature. Once the wax/Permethyl mixture is cooled to room temperature, the Ronasphere/DC245 pre-mix, and the propylparabens, and tocopherol acetate are added, and the combined mixture is milled using a Turrax T25 on low speed (about 8000 rpms) until homogeneous to form the lightly colored phase. Next, the clear water phase and the colored Ronasphere phase are combined and milled using a Turrax T25 on low speed (about 8000 rpms) until the water is full incorporated and an emulsion is formed. The resulting composition then is incorporated into appropriate packaging.

### Example 4

A liquid foundation of the present invention is prepared as follows:

| Ingredient | Wt % |
|---|---|
| DC9040 Cross Linked Elastomer Gel¹ | 44.25 |
| Elastomer Gel (KSG 21)² | 5.00 |
| Cyclomethicone (DC245) | 10.00 |
| Vinyl Block Copolymer | 2.00 |
| Iron Oxides-silicone coated | 4.00 |
| Titanium Dioxide-silicone coated | 8.00 |
| Propylparabens | 0.25 |
| Tocopherol Acetate | 0.50 |
| Isoeicosane (Permethyl 102A) | 5.00 |
| Alkyl Methicone (DC AMS C30 wax) | 1.50 |
| Water | 9.35 |
| Glycerin | 7.00 |
| Niacinamide | 2.00 |
| Panthenol | 0.50 |
| Sodium Dehydroacetate | 0.30 |
| Disodium EDTA | 0.10 |
| Phenoxyethanol | 0.25 |

¹12% Dimethicone/Vinyl Dimethicone crosspolymer (aver. particle size of at least 20 microns) in cyclomethicone;
²25% Dimethicone/Copolyol Crosspolymer in dimethicone

In a suitable vessel, the water, glycerine, niacinamide, panthenol, sodium dehydroacetate, disodium EDTA and phenoxyethanol are added and mixed using conventional technology until a clear water phase is achieved. In a separate vessel, the AMS wax and Permethyl are added and heated to 75° C. with gentle mixing. In a third vessel, the vinyl block copolymer are added followed by the iron oxides, titanium dioxide, then milled using high shear (about 20,000 units) to deagglomerate the pigments, forming an iron oxides/titanium dioxide/DC245 pre-mix. Once the wax/Permethyl mixture is fully molten, the DC9040 and KSG21 elastomers are added to this mixture and mixed until homogeneous. The wax/Permethyl/elastomer mixture is mixed using a Heidolph overhead stirrer (model #RZR50), or equivalent, on low speed (about 50-100 rpm) while cooling the mixture to room temperature. Once wax/Permethyl/elastomer mixture is cooled to room temperature, the propylparabens, tocopherol acetate, and the iron oxides/titanium dioxide/DC245 pre-mix are added, and the combined mixture is milled using a Turrax T25 on low speed (about 8000 rpm) until homogeneous to form the colored phase. Next, the clear water phase and the colored phase are combined, then milled using a Turrax T25 on low speed (about 8000 rpm) until the water is fully incorporated and an emulsion is formed. The resulting composition is incorporated into appropriate packaging.

### Example 5

A liquid make-up with SPF is made by mixing the following ingredients as detailed below.

| Ingredient | Wt % |
|---|---|
| Non-emulsifying Elastomer Gel (DC9040) | 40.00 |
| Emulsifying Elastomer Gel (KSG 21) | 5.00 |
| Vinyl Block Copolymer | 0.50 |
| Cyclomethicone (DC245) | 13.63 |
| Isoeicosane (Permethyl 102A) | 4.50 |
| Titanium Dioxide | 8.00 |
| Iron Oxides | 2.50 |
| Butylmethoxydibenzoylmethane | 1.00 |
| Octylsalicylate | 2.00 |
| Octocrylene | 0.50 |
| Propylparabens | 0.10 |
| Ethylparabens | 0.20 |
| Tocopherol Acetate | 0.50 |
| Water | 10.00 |
| Glycerin | 7.00 |
| Niacinamide | 2.00 |
| Phenylbenzimidazole Sulphonic Acid (Parsol | 1.00 |
| HS) | |
| Triethanolamine | 0.62 |
| Panthenol | 0.50 |
| Methyl parabens | 0.10 |
| Disodium EDTA | 0.10 |
| Benzyl alcohol | 0.25 |

In a suitable vessel, add the triethanolamine and Parsol HS to the water, then mix using conventional technology until a clear phase is achieved. Then add the water, glycerin, niacinamide, panthenol, methyl parabens, disodium EDTA, and benzyl alcohol. They are mixed using the same system until a clear phase is achieved. In a separate vessel, pre-disperse the vinyl block copolymer in the cyclomethicone then add the iron and titanium dioxides and mill to the desired particle size. Then add propyl parabens, ethyl parabens, KSG21, and Permethyl with high shear mixing e.g., Standard Silverson assembly at 8,000 rpm. Then, add the DC9040 and tocopherol acetate and mill using a Silverson on low speed until homogeneous. In a separate vessel, warm and agitate the butylmethoxydibenzoylmethane, octylsalicylate and octocrylene until a clear uniform liquid is obtained. Add this liquid to the pigment/elastomer dispersion and mix with a Silverson on low speed until homogeneous. Then, add the clear water phase to the colored phase and mill again using a Silverson at 8,000 rpm until the water is fully incorporated and an emulsion is formed. The resulting composition is incorporated into appropriate packaging.

### Examples 6-7

Cream foundations are made that are suitable for application to the face to provide softening, moisturization and conditioning and effective reduction in the appearance of oily/shiny skin.

| Ingredient | 6 | 7 |
|---|---|---|
| Cyclomethicone | 31.00 | 31.00 |
| DC9040 Silicone Elastomer Gel | 15.50 | 15.50 |
| Ethylene/Acrylic Acid Copolymer microspheres | 10.00 | 10.00 |
| (Flobeads EA 209 supplied by Kobo Products Inc.) | | |
| Emulsifying Elastomer Gel (KSG 210) | 5.00 | 5.00 |
| Silica and Titanium Dioxide and Iron Oxides | 10.00 | — |
| (Ronasphere LDP) | | |
| Vinyl Block Copolymer | 2.00 | 2.00 |
| Magnesium Aluminum Silicate (Sebumase) | — | 5.00 |
| Silica | — | 5.00 |
| Dimethicone Copolyol (Abil EM90) | 0.50 | 0.50 |
| Water | 15.00 | 15.00 |
| Glycerin | 10.00 | 10.00 |
| Niacinamide | 3.50 | 3.50 |
| Preservative | 0.50 | 0.50 |
| Total | 100.00 | 100.00 |

In a suitable vessel, mix the cyclomethicone and vinyl block copolymer until dissolved. Then, add the Flobeads, Ronasphere particles, Sebumase, and silica, and mill until uniformily dispersed and desired particle size is achieved. Next, add the Abil EM90, KSG-210, and DC9040, and mix under high shear. Admix the water, glycerin, niacinamide, and preservative materials in a separate vessel until clear. Emulsify the mixture by adding the water phase mixture to the silicone mixture while mixing under high shear.

### Examples 8-10

Cream foundations are made that are suitable for application to the face to provide a long-lasting, softening, moisturization, and conditioning effect, and to reduce the appearance of oily/shiny skin.

| | | 8 | 9 | 10 |
|---|---|---|---|---|
| Ingredient | Phase | Wt % | Wt % | Wt % |
| Silicone Phase | | | | |
| Non-emulsifying Elastomer Gel (DC9040) | A | 40 | 25 | 30 |
| Cyclopentasiloxane (DC245) | A | 18 | 32 | 12 |
| Emulsifying Elastomer Gel (KSG210) | A | 5 | 5 | 5 |
| Allyl Methacrylates Crosspolymer | A | — | — | 1.5 |
| Treated Powders¹ | A | 11 | 12 | 12.5 |
| Vinyl Block Copolymer | A | 1 | 1 | 1 |
| Polysilicone 7 (silicones plus polymer | A | — | — | 17 |
| SA70) | | | | |
| Solidifying agents | | | | |
| Stearyl Dimethicone | | — | 3 | — |
| Water Phase | | | | |
| Deionized Water | B | 9 | 10 | 16 |
| Glycerin | B | 10 | 10 | 3 |
| Preservatives | B | 1 | 1 | 1 |
| Sunscreens | A | 4 | — | — |

¹mixture of iron oxides and titanium dioxides

In a suitable stainless steel vessel, mill the phase A ingredients until homogeneous and desired particle size is achieved. In a separate vessel equipped with a heat source, heat the water phase (B) materials to 50° C. and mix until homogeneous. If using solidifying agents, heat the cyclopentasiloxane mixture to a temperature required to melt the solidifying agents and add the solidifying agents. Cool both the water phase and silicone phase to below 30 C and mix under high shear to form an emulsion.

### Example 11

Lip Cosmetic Product

| | |
|---|---|
| Ingredients | Weight (%) |

Group A:

| | |
|---|---|
| Silicone Gum¹ | 12.60 |
| Isododecane² | 12.10 |

Group B:

| | |
|---|---|
| Isododecane² | 43.38 |
| Vinyl Block Copolymer | 0.50 |
| Bentonite Clay⁴ | 1.00 |
| Propylene Carbonate | 0.32 |
| Red #6 Calcium Lake | 1.00 |
| Red #7 Barium Lake | 3.00 |
| Titanium Dioxide | 1.50 |
| Mica | 2.20 |
| Organosiloxane Resin³ | 22.40 |

¹2,500,000 cSt Dimethicone Gum available as SE 63 from General Electric.
²Permethyl 99A available from Permethyl Corp.
³MQ Resin (0.7:1 ratio M:Q) available as 1170-002 from General Electric.
⁴Bentone 38 available from Rheox.

Combine Group A ingredients together in a beaker and mix with a propeller mixer until uniform. Combine all Group B ingredients except the propylene carbonate, and hand-mix to roughly incorporate the dry powders. Homogenize the entire formulation using a Ross ME 100 LC homogenizer at about 7500 rpm until all pigments are fully dispersed. Next, while continuing the homogenization process, slowly add the propylene carbonate until mixture thickens. Combine Group A mixture with Group B mixture in a beaker and mix with a propeller mixer until uniform. Transfer the resulting fluid to individual packages.

### Example 12

### Liquid Foundation Cosmetic Product

| | |
|---|---|
| Ingredients | Weight (%) |

Group A:

| | |
|---|---|
| Organosiloxane Resin¹ | 4.48 |
| Cyclomethicone² | 10.11 |
| Vinyl Block Copolymer | 1.00 |
| Silicone-polyether Emulsifier³ | 10.00 |

Group B:

| | |
|---|---|
| Silicone-Treated Titanium Dioxide | 6.50 |
| Silicone-Treated Yellow Iron Oxide | 0.28 |
| Silicone-Treated Red Iron Oxide | 0.15 |
| Silicone-Treated Black Iron Oxide | 0.06 |

Group C:

| | |
|---|---|
| 2,500,000 cSt Silicone Gum⁴ | 2.52 |
| Cyclomethicone² | 4.90 |

Group D:

| | |
|---|---|
| Water | 49.50 |
| Glycerin | 10.00 |
| Methyl Paraben | 0.20 |
| 2-Phenoxyethanol | 0.30 |

¹MQ Resin available as 1170-002 from General Electric.
²Cyclomethicone available as 245 fluid from Dow Corning.
³Silicone-Polyether Emulsifier available as DC3225C from Dow Corning.
⁴Dimethicone Gum (2,500,000 cSt) available as SE63 from General Electric.

Combine Group A and Group B ingredients, and homogenize at 9500 rpm for 15 minutes. Add Group C ingredients and homogenize at 2000 rpm for 2 minutes. Combine Group D ingredients in a separate container and mix with a propeller mixer until a clear solution forms. Add the Group D solution to the mixture of Groups A, B, and C very slowly while homogenizing at 2000 rpm. When all of the Group D solution has been incorporated, homogenize the entire mixture at 2000 rpm for an additional 10 minutes. Finally, homogenize the entire mixture at 5000 rpm for 5 minutes. Transfer the resulting fluid to individual packages.

### Example 13

### Mascara Cosmetic Product

| | |
|---|---|
| Ingredients | Weight (%) |

Group A:

| | |
|---|---|
| Organosiloxane Resin¹ | 9.60 |
| Cyclomethicone² | 8.32 |
| Vinyl Block Copolymer | 1.50 |
| Silicone-polyether Emulsifler³ | 10.00 |

Group B:

| | |
|---|---|
| Silicone-Treated Black Iron Oxide | 5.00 |

Group C:

| | |
|---|---|
| 2,500,000 cSt Silicone Gum⁴ | 5.40 |
| Cyclomethicone² | 15.19 |

Group D:

| | |
|---|---|
| Water | 43.50 |
| Sodium Chloride | 1.00 |
| Methyl Paraben | 0.20 |
| 2-Phenoxyethanol | 0.30 |

¹MQ Resin available as 1170-002 from General Electric.
²Cyclomethicone available as 244 fluid from Dow Corning.
³Silicone-Polyether Emulsifier available as DC3225C from Dow Corning.
⁴Dimethicone Gum (2,500,000 cSt) available as SE63 from General Electric.

Combine Group A and Group B ingredients together and homogenize at 9500 rpm for 15 minutes. Add Group C ingredients and homogenize at 2000 rpm for 2 minutes. Combine Group D ingredients in a separate container and mix with a propeller mixer until a clear solution forms. Add the Group D solution to the mixture of Groups A, B, and C very slowly while homogenizing at 2000 rpm. When all of the Group D solution has been incorporated, homogenize the entire mixture at 2000 rpm for an additional 10 minutes. Finally, homogenize the entire mixture at 5000 rpm for 5 minutes. Transfer the resulting fluid to individual packages.

### Example 14

### Liquid Foundation

| | | |
|---|---|---|
| A | Dimethicone Copolyol | 2.20 |
| B | Cyclomethicone | 27.26 |
| B | Vinyl Block Copolymer | 1.25 |
| B | Titanium Dioxide | 8.25 |
| B | Yellow Iron Oxide | 1.19 |
| B | Red Iron Oxide | 0.53 |
| B | Black Iron Oxide | 0.11 |
| B | Talc | 3.46 |
| B | Titanium Dioxide, Stearic Acid, Al, Tri-K-MT-100T | 0.25 |
| B | Mica, BaSO4, TiO2(50:30:20), Kobo Low Luster pigment | 0.10 |
| B | Silicone 350 CS, 200 fluid | 2.00 |
| B | Silicone 50 CS, 200 fluid | 3.00 |
| B | Silica, Presperse Spheron L-1500 | 0.10 |
| C | Polyethylene wax | 0.40 |
| D | Trihydroxystearin, Thixin R | 0.30 |
| D | Cyclomethicone | 1.00 |
| E | Propylparaben | 0.25 |
| E | Fragrance | 0.05 |
| F | Propylene Glycol | 8.00 |
| F | Methylparaben | 0.12 |
| F | Water | 36.88 |
| F | Sodium Chloride crystal | 2.00 |
| F | Sodium Dehydroacetate | 0.30 |
| F | PVP K-17, BASF Luviskol K17 Powder | 1.00 |
| | TOTAL | 100.00 |

Dissolve vinyl block copolymer in the silicone (cyclomethicone+silicone fluids). Add the remaining pigments and particles in Phase B, and high shear mill until desired particle size. Add Phase A component to Phase B and continue mixing at low shear. Heat until temperature reaches 75-85° C. Add phase C and mix until melted. In a separate container mix Phase D. Cool batch to 50-65° C., and add phase D and Phase E ingredients. Reduce temperature to 20-30° C. while mixing and add ingredients in Phase F. Mix until uniform and transfer to appropriate container.

What is claimed is:

1. A stable dispersion comprising
(a) about 0.5% to about 85%, by weight, of solid particles;
(b) about 0.01% to about 60%, by weight, of an vinyl copolymer; and
(c) about 10% to about 90%, by weight, of a hydrophobic solvent.

2. The dispersion of claim 1 wherein the solid particles have a number weighted average particle size of about 10 nm to about 100 um.

3. The dispersion of claim 1 wherein the solid particles comprise an organic pigment, an inorganic pigment, or a mixture thereof.

4. The dispersion of claim 3 wherein the pigment comprises a metal oxide, a lake dye, a toner, or a mixture thereof.

5. The dispersion of claim 1 wherein the solid particle comprises an active sunscreen compound, an ultraviolet light absorber, or a mixture thereof.

6. The dispersion of claim 1 wherein the solid particle comprises an opacifier, a skin care active, a hair care active, a nail care active, hair and skin feel modifier or a mixture thereof.

7. The dispersion of claim 1 wherein the vinyl copolymer comprises monomers B and C and optional monomer A, wherein monomer A, when present, is at least one free radically polymerizable vinyl monomer; monomer B comprises at least one reinforcing monomer copolymerizable with A and C and is selected from the group consisting of polar monomers and macromers having a Tg or a Tm above about −20° C.; and monomer C has a general formula X(Y)ₙSi(R)₃₋ₘZₘ, wherein X is a vinyl group copolymerizable with the A and B monomers; Y is a divalent linking group; R is hydrogen, lower alkyl, aryl, or alkoxy; Z is a monovalent siloxane polymeric moiety having a number average molecular weight of at least about 500, is essentially unreactive under copolymerization conditions, and is pendant from the vinyl polymeric backbone; n is 0 or 1; and m is an integer from 1 to 3.

8. The dispersion of claim 7 wherein monomer C has a weight average molecular weight of from about 1,000 to about 50,000.

9. The dispersion of claim 7 wherein the monomer C has a formula selected from the following group:

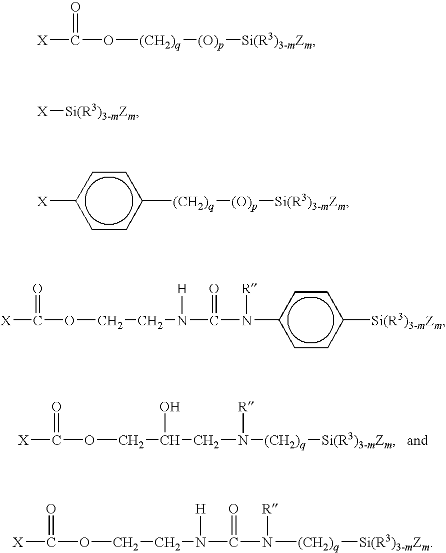

10. The dispersion of claim 7 wherein the vinyl copolymer comprises 0% to about 98% of monomer A, about 0.1% to about 98% of monomer B, and about 0.1% to about 50% of monomer C.

11. The dispersion of claim 10 wherein a combination of the B and C monomers comprises about 50% to about 99.9% of the total monomers in the copolymer.

12. The dispersion of claim 7 wherein the vinyl copolymer comprises a plurality of amino groups or a plurality of carboxyl groups.

13. The dispersion of claim 1 wherein the vinyl copolymer has a structure: 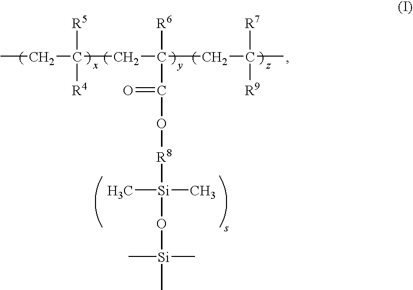
wherein R⁴ is selected from the group consisting of —C(═O)R^{a}, —C(═O)OR^{a}, and —C(═O)NR^{a}R^{b};
R⁵ is selected from the group consisting of C₁₋₃alkyl, aryl, and —C(═O)R^{a};
R⁶ is selected from the group consisting of C₁₋₃alkyl, aryl, and —C(═O)R^{a};
R⁷ is selected from the group consisting of C₁₋₃alkyl, aryl, and —C(═O)R^{a};
R⁸ is CH₂₁₋₆;
R⁹, independently, is selected from the group consisting of carboxyl, carboxylic acid ester, hydroxyl, sulfonate, sulfate, phosphate, phosphonate, nitro, carbohydrate, quaternary ammonium salt, phosphate ester, carbonyl, amino, amide, imide, an aryl group, a heteroaryl group, an aliphatic hydrocarbon, an aliphatic heterocyclic group, polyethylene oxide, polypropylene oxide, silicone, fluorocarbon, polyester, and urethane;
s is an integer from 1 to 500;
R^{a} and R^{b}, independently, are C₁₋₁₂ alkyl or aryl;
x is an integer 0 to 500;
y is an integer 2 to 25; and
z is an integer 1 to 500.

14. The dispersion of claim 13 wherein R⁴ is —C(═O)R^{a}; R⁵, R⁶, and R⁷, independently, are C₁₋₃alkyl, aryl, and —C(═O)R^{a}; R⁸ is CH₂₁₋₆; R⁹ is amino or carboxy; and R^{a} and R^{b} are C₁₋₃alkyl.

15. The dispersion of claim 13 wherein in the vinyl copolymer has a structure:

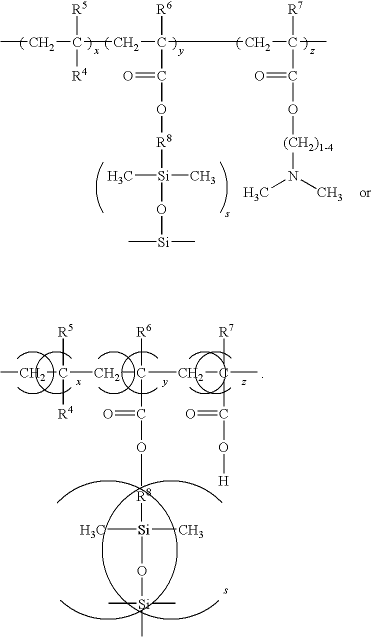

16. The dispersion of claim 1 wherein the vinyl copolymer has a structural formula

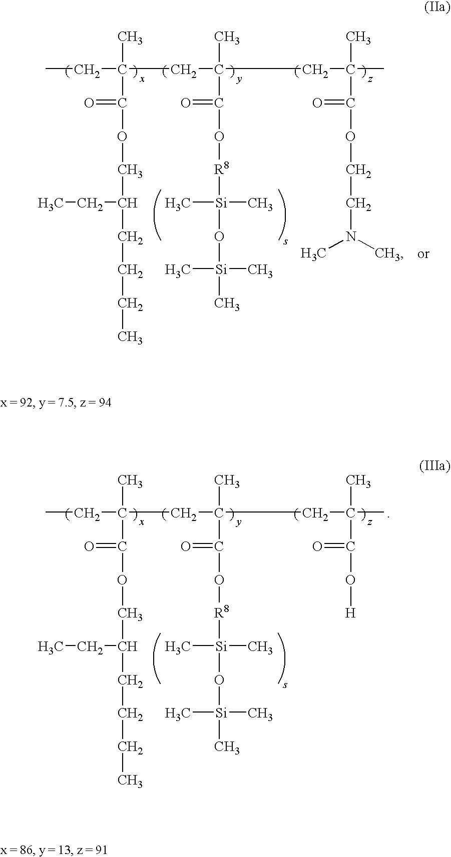

17. The dispersion of claim 1 wherein the vinyl copolymer is selected from the group consisting of acrylic acid/n-butyl methacrylate/polydimethylsiloxane (PDMS) macromer—20,000 molecular weight (10/70/20 w/w/w), N,N-dimethylacrylamide/isobutyl methacrylate/PDMS macromer—20,000 molecular weight (20/60/20 w/w/w), dimethylaminoethyl methacrylate/isobutyl methacrylate/2-ethylhexyl methacrylate/PDMS macromer—20,000 molecular weight (25/40/15/20 w/w/w/w), dimethylacrylamide/PDMS macromer—20,000 molecular weight (80/20 w/w), t-butyl acrylate/t-butyl methacrylate/PDMS macromer—10,000 molecular weight (56/24/20 w/w/w), t-butyl acrylate/PDMS macromer—10,000 molecular weight (80/20 w/w), t-butyl acrylate/N,N-dimethylacrylamide/PDMS macromer—10,000 molecular weight (70/10/20 w/w/w), t-butyl acrylate/acrylic acid/PDMS macromer—10,000 molecular weight (75/5/20 w/w/w), N,N-dimethylaminoethyl methacrylate/isobutyl methacrylate/PDMS macromer—20,000 molecular weight (10/70/20 w/w/w), quaternized N,N-dimethylaminoethyl methacrylate/isobutyl methacrylate/PDMS macromer—20,000 molecular weight (60/20/20 w/w/w) (MW—500,000), acrylic acid/methyl methacrylate/PDMS macromer—20,000 molecular weight (40/40/20 w/w/w), acrylic acid/n-butyl methacrylate/PDMS macromer—20,000 molecular weight (10/70/20 w/w/w), acrylic acid/isopropyl methacrylate/PDMS macromer—10,000 molecular weight (25/65/10 w/w/w), N,N-dimethylacrylamide/methoxyethyl methacrylate/PDMS macromer—20,000 molecular weight (60/25/15 w/w/w) (MW—200,000), N,N-dimethylacrylamide/isobutyl methacrylate/2-ethylhexyl methacrylate/PDMS macromer—20,000 molecular weight (12/64/4/20 w/w/w), N,N-dimethylaminoethyl methacrylate/isobutyl methacrylate/2-ethylhexyl methacrylate/PDMS macromer—20,000 molecular weight (30/40/10/20 w/w/w/w), and t-butyl acrylate/PDMS macromer—10,000 molecular weight (80/20 w/w).

18. The dispersion of claim 1 wherein the hydrophobic solvent has a solubility parameter (γ) of about 8 or less.

19. The dispersion of claim 1 wherein the hydrophobic solvent is selected from the group consisting of dimethicone, cyclomethicone, a silicone fluid, mineral oil, and mixtures thereof.

20. A method of preparing a stable dispersion of solid particles in a hydrophobic solvent comprising admixing a vinyl copolymer with the solid particles and the hydrophobic solvent.

21. A personal care composition comprising a dispersion of claim 1.

22. A cosmetic composition comprising a dispersion of claim 1.
